(19) United States
(12) Patent Application Publication
(10) Pub. No.: US 2004/0053412 A1
(43) Pub. Date: Mar. 18, 2004

(54) Cells resistant to toxic genes and uses thereof

(75) Inventors: James L. Hartley, Frederick, MD (US); Michael A. Brasch, Gaithersburg, MD (US); Gary F. Temple, Washington Grove, MD (US)

Correspondence Address: STERNE, KESSLER, GOLDSTEIN & FOX PLLC, 1100 NEW YORK AVENUE, N.W., WASHINGTON, DC, 20005, US

(73) Assignee: Invitrogen Corporation

(21) Appl. No.: 10/396,696

(22) Filed: Mar. 26, 2003

Related U.S. Application Data
(63) Continuation of application No. 09/518,188, filed on Mar. 2, 2000, now abandoned.
(60) Provisional application No. 60/122,392, filed on Mar. 2, 1999.

(51) Int. Cl. C12N 15/74; C12N 1/21

(52) U.S. Cl. 435/473; 435/252.33

(57) ABSTRACT

The present invention relates to cells and cell strains that are resistant to the killing effects of one or more toxic genes, particularly those that kill hosts in the absence of a suppressing function, e.g., kicB or ccdB. The host cells may comprise one or more suppression mutations, such as deletional or insertional mutations in gyrA, endA, or recA, or combinations thereof (particularly gyrA/endA or gyrA/recA), which allow cell strains carrying the one or more suppression mutations to survive the presence and/or expression of one or more toxic genes within their genome or in extrachromosomal genetic elements within the host cell. Preferred host cell strains include prokaryotic host cells, particularly specified strains of E. coli containing the gyrA462 mutation and/or one or more additional mutations, such as DB3, DB3.1, DB4 and DB5. The host cells of the invention are useful in producing recombinant genetic constructs, particularly cDNAs and cDNA libraries, via traditional genetic engineering techniques or via recombinational cloning using engineered recombination sites. The host cells are also useful in cloning and propagation of toxic genes that act upon DNA gyrase, such as ccdB.

### CROSS REFERENCE TO RELATED APPLICATIONS

The present application claims the benefit of the filing date of U.S. Provisional Application No. 60/122,392, filed Mar. 2, 1999. The present application is also related to U.S. application Ser. No. 08/486,139, filed Jun. 7, 1995 (now abandoned), Ser. No. 08/663,002, filed Jun. 7, 1996 (now U.S. Pat. No. 5,888,732), Ser. No. 09/005,476, filed Jan. 12, 1998, Ser. No. 09/177,387, filed Oct. 23, 1998, Ser. No. 09/233,492, filed Jan. 20, 1999, Ser. No. 09/233,493, filed Jan. 20, 1999, No. 60/122,389, filed Mar. 2, 1999, No. 60/126,049, filed Mar. 23, 1999, No. 60/136,744, filed May 28, 1999, Ser. No. 09/296,280, filed Apr. 22, 1999, Ser. No. 09/296,281, filed Apr. 22, 1999, Ser. No. 09/432,085, filed Nov. 2, 1999, and Ser. No. 09/438,358, filed Nov. 12, 1999. The disclosures of all of the applications cross-referenced above are incorporated by reference herein in their entireties.

### BACKGROUND OF THE INVENTION

1. Field of the Invention

The present invention relates generally to cell and molecular biology. More particularly, the present invention relates to mutant host cell strains that are resistant to the effects of the expression of one or more toxic genes. Most particularly, the invention relates to such host cell strains carrying one or more mutations in their DNA gyrase gene which renders the host cell strains resistant to the effects of toxic genes that act upon DNA gyrase. The host cell strains of the invention are useful for a variety of purposes, including but not limited to amplification and cloning of nucleic acid molecules by recombinational cloning methods, and for cloning and propagation of toxic genes.

2. Related Art

Toxic Genes

The genes ccdA and ccdB are the antidote and toxin genes respectively of the E. coli F plasmid (P. Bernard, et al., J. Mol. Biol. 234: 534 (1993)). Together, they ensure the death of daughter cells that do not receive a copy of F. Expression of the ccdB protein interferes with the rejoining step of DNA gyrase, causing the host cell chromosome to be cut to pieces. Plasmids that contain the ccdB gene without the antidote gene can be propagated in a gyrase mutant host cell strain, such as E. coli gyrA462 (T. Mike, et al., J. Mol. Biol. 225: 39 (1992)). Other toxic genes have also been identified, for example ΦX E which is toxic when expressed in E coli unless the host cell lacks an slyD gene which encodes cis-trans peptidyl-prolyl isomerase upon which the ΦXE gene acts (Liu, Q. et al., Curr. Biol. 8:1300-1309 (1998)).

For certain applications, such as cloning of nucleic acid molecules by recombinational cloning techniques like those described herein, and for cloning and propagation of genetic constructs containing one or more toxic genes, it would be advantageous to have a choice of mutant host cell strains that are resistant to the effects of toxic genes such as ccdB which is used in preferred recombinational cloning methods. The present invention provides such mutant host cell strains.

### SUMMARY OF THE INVENTION

The present invention relates generally to mutant host cells and host cell strains that are resistant to the effects of the expression of one or more toxic genes. Most particularly, the invention relates to such host cells and host cell strains carrying one or more mutations, particularly in their DNA gyrase gene, which renders the host cells and host cell strains resistant to the effects of the expression of one or more toxic genes that act upon DNA gyrase. The invention also relates to host cells and host cell strains having one or more mutations which allow the host cell to grow in the presence of a toxic gene selected from the group consisting of ccdB, kicB, DpnI, ΦX E, and the like.

Thus, in one aspect the invention provides mutant host cells, which may be Escherichia coli cells, containing a gyrA gene, an endA gene, and a recA gene, wherein the gyrA and endA genes contain one or more mutations that render the host cell resistant to the expression of one or more toxic genes including, but not limited to, toxic genes such as ccdB, kicB, DpnI, an apoptosis-related gene, a retroviral gene, a defensin, a bacteriophage lytic gene, an antibiotic sensitivity gene, an antimicrobial sensitivity gene, a plasmid killer gene, and a eukaryotic transcriptional vector gene that produces a gene product toxic to bacteria, and most particularly ccdB. The invention also provides such mutant host cells which further comprise one or more mutations in the recA gene (including, but not limited to, Δ(srl-recA) 1398), and/or one or more genetic elements (including, but not limited to, a tetracycline resistance gene or transposon Tn10). The invention also relates to such mutant host cells which comprise one or more additional mutations, such as mutations in recA, endA, mcrA, mcrB, mcrC, hsd, deoR, and the like, preferably in recA or endA or more preferably in both recA and endA. Preferred mutations in the gyrA gene according to this aspect of the invention include gyrA462. While the invention relates to any mutant host cell or host cell strain having the features and characteristics described herein, preferred such host cells and host cell strains include, but are not limited to, a DB3 cell (deposit number NRRL B-30097), a DB3.1 cell (deposit number NRRL B-30098), a DB4 cell (deposit number NRRL B-30106), and a DB5 cell (deposit number NRRL B-30107), or a mutant or derivative thereof.

The invention also relates to host cell strains containing a mutation in the DNA gyrase gene (such as those described herein) and further containing one or more additional mutations in one or more genes selected from the group consisting of recA, endA, mcrA, mcrB, mcrC, hsd, deoR, and the like, preferably in recA or endA or more preferably in both recA and endA. Such host cell strains are useful in cloning one or more nucleic acid molecules (e.g., one or more genes) of interest, and host cell strains containing mutations in at least two genes that make them resistant to the activities of two or more toxic genes are useful, for example, in cloning two or more genes, for example by recombinational cloning methods in which the two nucleic acid molecules of interest are contained on one or more genetic constructs (e.g. a vector) that has two toxic genes, such that the host cell must be resistant to both toxic genes in order to grow and express (or replicate) the two or more genes of interest.

In another aspect, the invention relates to methods of cloning a genetic construct comprising one or more toxic genes, such as those toxic genes described above. Methods according to this aspect of the invention preferably comprise introducing a genetic construct comprising one or more toxic genes into one or more of the host cells or host cell strains of the invention, and cultivating the host cell or host cell strain under conditions favoring the clonal expansion of the host cell.

In another aspect, the invention relates to kits comprising one or more of the mutant host cells or mutant host cell strains of the invention. Kits according to this aspect of the invention may comprise one or more of the host cells or host cell strains of the invention, and may further comprise one or more additional components suitable for use with, or for cultivation of, the host cells or host cell strains of the invention. Such additional components may include, for example, one or more culture media suitable for cultivation of the host cells or host cell strains of the invention, one or more selection agents (such as one or more antibiotics, dyes, detergents, antimicrobial agents, and the like), one or more genetic constructs comprising one or more toxic genes (such as a vector comprising one or more of the toxic genes described herein, most preferably ccdB), one or more buffers, and the like.

Other preferred embodiments of the present invention will be apparent to one of ordinary skill in light of what is known in the art, in light of the following drawings and description of the invention, and in light of the claims.

### BRIEF DESCRIPTION OF THE DRAWINGS

FIG. 1 is a schematic depiction of an Entry Vector, containing the ccdB toxin gene flanked by recombination sites attL1 and attL2, and a kanamycin resistance (Kan^{r}) gene.

FIG. 2 is a schematic depiction of a Destination Vector, containing the ccdB toxin gene and an inactive ccdA antidote gene, flanked by recombination sites attR1 and attR2, and an ampicillin resistance (amp^{r}) gene.

FIG. 3 is a depiction of the cloning sites of the Entry Vector pENTR-7, showing the location of the ccdB gene in relation to the flanking attL1 and attL2 recombination sites and the multiple cloning sites contained in this vector.

FIG. 4 is a restriction map of the Destination Vector pTrc-DEST1, showing the location of the ccdA and ccdB genes in relation to the flanking attR1 and attR2 recombination sites, the ampicillin resistance gene, and the multiple cloning sites contained in this vector.

FIG. 5 is a schematic depiction of recombinational cloning, using vectors carrying the ccdB gene.

### DETAILED DESCRIPTION OF THE INVENTION

Definitions

In the description that follows, a number of terms used in molecular and cellular biology are utilized extensively. In order to provide a clear and consistent understanding of the specification and claims, including the scope to be given such terms, the following definitions are provided.

Host cell: A “host” or “cell” as these terms are used herein, and which terms may be used interchangeably with each other and with the terms “host cell” and “host cell strain,” includes prokaryotic or eukaryotic organisms that can be genetically engineered. Typical prokaryotic host cells that may be used in accordance with the present invention include, but are not limited to, bacterial cells such as those of the genera Escherichia spp. (particularly E. coli), Streptomyces spp., Erwinia spp., Klebsiella spp., Bacillus spp. (particularly B. cereus, B. sublilis, and B. megaterium), Serratia spp., Pseudomonas spp. (particularly P. aeruginosa) and Salmonella spp. (particularly S. typhi or S. typhimurium). It will be understood, of course, that there are many suitable strains and serotypes of each of the host cell species described herein, any and all of which may be used in accordance with the invention. Preferred as a host cell is E. coli, and particularly preferred are E. coli strains RR1 (E. coli F-mcrB mrr hsdS20(r_{B}- m_{B}-) recA13 leuB6 ara-14 proA2 lacY1 galK2 xyl-5 mtl-1 rpsL20(Sm^{r}) supE44 λ-), DH10B (E. coli F- mcrA Δ(mrr-hsdRMS-mcrBC) Φ80dlacZΔM15 ΔlacX74 endA1 recA1 deoR Δ(ara, leu)7697 araD139 galU galK nupG rpsL λ-), Stbl2 (E. coli mcrA (mcrBC-hsdRMS-mrr)endA1 recA1thigyrA96relA1supE44(lac-proAB) λ-) DH5α, and BL21Si, which are available commercially (Life Technologies, Inc; Rockville, Md.). Typical eukaryotic host cells that may be used in accordance with the present invention include, but are not limited to, animal cells (particularly mammalian (including human), avian, amphibian, reptilian, nematode and insect cells), plant cells, and fungal (including yeast) cells. For examples of these and other suitable hosts, see Maniatis et al, Molecular Cloning: A Laboratory Manual, Cold Spring Harbor Laboratory, Cold Spring Harbor, N.Y. (1982).

Recombinational Cloning: is a method described herein and in U.S. application Ser. No. 08/486,139, filed Jun. 7, 1995 (now abandoned), Ser. No. 08/663,002, filed Jun. 7, 1996 (now U.S. Pat. No. 5,888,732), Ser. No. 09/005,476, filed Jan. 12, 1998, Ser. No. 09/177,387, filed Oct. 23, 1998, Ser. No. 09/233,492, filed Jan. 20, 1999, Ser. No. 09/233,493, filed Jan. 20, 1999, No. 60/122,389, filed Mar. 2, 1999, No. 60/126,049, filed Mar. 23, 1999, No. 60/136,744, filed May 28, 1999, Ser. No. 09/296,280, filed Apr. 22, 1999, Ser. No. 09/296,281, filed Apr. 22, 1999, Ser. No. 09/432,085, filed Nov. 2, 1999, and Ser. No. 09/438,358, filed Nov. 12, 1999, the disclosures of all of which are incorporated herein by reference in their entireties. In the recombinational cloning process, segments of nucleic acid molecules or populations of such molecules are fused, exchanged, inserted, replaced, substituted or modified, in vitro or in vivo.

Selectable marker: is a DNA segment that allows one to select for or against a molecule (e.g., a replicon) or a cell that contains it, often under particular conditions. These markers can encode an activity, such as, but not limited to, production of RNA, peptide, or protein, or can provide a binding site for RNA, peptides, proteins, inorganic and organic compounds or compositions and the like. Examples of Selectable markers include but are not limited to: (1) DNA segments that encode products which provide resistance against otherwise toxic compounds (e.g., antibiotics); (2) DNA segments that encode products which are otherwise lacking in the recipient cell (e.g., tRNA genes, auxotrophic markers); (3) DNA segments that encode products which suppress the activity of a gene product or a functional site; (4) DNA segments that encode products which can be readily identified (e.g., phenotypic markers such as P-galactosidase, green fluorescent protein (GFP), and cell surface proteins); (5) DNA segments that bind products which are otherwise detrimental to cell survival and/or function; (6) DNA segments that otherwise inhibit the activity of any of the DNA segments described in Nos. 1-5 above (e.g., antisense oligonucleotides); (7) DNA segments that bind products that modify a substrate (e.g. restriction endonucleases); (8) DNA segments that can be used to isolate or identify a desired molecule (e.g. specific protein binding sites); (9) DNA segments that encode a specific nucleotide sequence which can be otherwise non-functional (e.g., for PCR amplification of subpopulations of molecules); (10) DNA segments, which when absent, directly or indirectly confer resistance or sensitivity to particular compounds; and/or (11) DNA segments that encode products which are toxic in recipient cells.

Toxic gene. A toxic gene can be a DNA that is expressed as a toxic gene product (a toxic protein or RNA), or can be toxic in and of itself (In the latter case, the toxic gene is understood to carry its classical definition of “heritable trait”.) Examples of such toxic gene products are well known in the art, and include, but are not limited to, restriction endonucleases (e.g., DpnI), apoptosis-related genes (e.g. ASK1 or members of the bcl-2/ced-9 family), retroviral genes including those of the human immunodeficiency virus (HIV), defensins such as NP-1, inverted repeats or paired palindromic DNA sequences, bacteriophage lytic genes such as those from ΦX174 (e.g., ΦX E) or bacteriophage T4; antibiotic sensitivity genes such as rpsL, antimicrobial sensitivity genes such as pheS, plasmid killer genes, eukaryotic transcriptional vector genes that produce a gene product toxic to bacteria, such as GATA-1, and genes that kill hosts in the absence of a suppressing function, e.g., kicB or ccdB. A toxic gene can alternatively be selectable in vitro, e.g., a restriction site.

Vector: is a nucleic acid molecule (preferably DNA) that provides a useful biological or biochemical property to an Insert. Examples include plasmids, phages, viruses, autonomously replicating sequences (ARS), centromeres, transposons, and other sequences which are able to replicate or be replicated in vitro or in a host cell, or to convey a desired nucleic acid segment to a desired location within a host cell. A vector can have one or more restriction endonuclease recognition sites at which the sequences can be cut in a determinable fashion without loss of an essential biological function of the vector, and into which a nucleic acid fragment can be spliced in order to bring about its replication and cloning. Vectors can further provide primer sites, e.g., for PCR, transcriptional and/or translational initiation and/or regulation sites, recombinational signals, replicons, Selectable markers, etc. Clearly, methods of inserting a desired nucleic acid fragment which do not require the use of homologous recombination, transpositions or restriction enzymes (such as, but not limited to, UDG cloning of PCR fragments (U.S. Pat. No. 5,334,575, entirely incorporated herein by reference), T:A cloning, and the like) can also be applied to clone a fragment into a cloning vector to be used according to the present invention. The cloning vector can further contain one or more selectable markers suitable for use in the identification of cells transformed with the cloning vector.

Other terms used in the fields of recombinant DNA technology and molecular and cell biology as used herein will be generally understood by one of ordinary skill in the applicable arts.

Host Cells

One aspect of the invention provides host cells and host cell strains that are resistant to the killing (bacteriocidal) or growth suppressive (bacteriostatic) activities of one or more toxic genes. Such host cells are useful in a variety of methods, including for example propagating nucleic acid molecules containing one or more toxic genes, and selection of host cells which have been successfully transformed with a genetic construct containing a gene of interest and a toxic gene.

A number of such selection schemes can be used with a variety of host cells, particularly E. coli cells and cell strains. One is to put a repressor gene on one segment of the subcloning plasmid, and a drug marker controlled by that repressor on the other segment of the same plasmid. Of course a way must exist for growing such a plasmid, i.e., there must exist circumstances under which the killer gene will not kill. There are a number of these genes known which require particular strains of E. coli. One such scheme is to use the restriction enzyme DpnI, which will not cleave unless its recognition sequence GATC is methylated. Many popular common E. coli strains methylate GATC sequences, but there are mutants in which cloned DpnI can be expressed without harm. Other restriction enzyme genes may also be used as a toxic gene for selection. In such cases, a host containing a gene encoding the corresponding methylase provides protected hosts for use in the invention. Similarly, the ccdB protein is a potent poison of DNA gyrase, efficiently trapping gyrase molecules in a cleavable complex, resulting in DNA strand breakage and cell death. Mutations in the gyrA subunit of DNA gyrase, specifically the gyrA462 mutation contained in the E. coli RR1 gyrA462 mutant designated DB1, confers resistance to ccdB (Bernard and Couturier, J. Mol. Biol. 226: 735-745 (1992)).

Hence, in one aspect the invention relates to mutant host cells and host cell strains that are resistant to the effects of the expression of one or more toxic genes. Host cells of this aspect of the invention may comprise one or more mutations in one or more genes within their genomes or on extrachromosomal or extragenomic DNA molecules (such as plasmids, phagemids, cosmids, etc.), including mutations in, for example, recA, endA, mcrA, mcrB, mcrC, hsd, deoR, and the like, preferably in recA or endA or more preferably in both recA and endA Most particularly, the invention relates to such host cells and host cell strains carrying one or more mutations, particularly in their DNA gyrase gene, which renders the host cells and host cell strains resistant to the effects of the expression of one or more toxic genes that act upon DNA gyrase.

In a first such aspect the invention provides mutant host cells, which may be Escherichia coli cells, containing a gyrA gene, an endA gene, and a r ecA gene, wherein the gyrA and endA genes contain one or more mutations that render the host cell resistant to the expression of one or more toxic genes. According to the invention, the one or more mutations may render the host cells and host cell strains resistant to toxic genes including, but not limited to, ccdB, kicB, DpnI, an apoptosis-related gene, a retroviral gene, a defensin, a bacteriophage lytic gene, an antibiotic sensitivity gene, an antimicrobial sensitivity gene, a plasmid killer gene, and a eukaryotic transcriptional vector gene that produces a gene product toxic to bacteria, and most particularly ccdB. The invention also provides such mutant host cells which further comprise one or more mutations in the recA gene (including, but not limited to, Δ(srl-recA)1398), and/or one or more genetic elements (including, but not limited to, a tetracycline resistance gene or transposon Tn10). Preferred mutations in the gyrA gene according to this aspect of the invention include gyrA462

One such host cell strain, E. coli strain DB2, has been constructed in accordance with the invention. DB2 cells contain the gyrA462 mutation and a mutation in endA. DB2 cells containing plasmids that express the ccdB gene (for example, Destination and Entry Vectors described below) are not killed by ccdB. This strain is available from Life Technologies and was deposited on Oct. 14, 1997, with the Collection, Agricultural Research Culture Collection (NRRL), 1815 North University Street, Peoria, Ill. 61604 USA as deposit number NRRL B-21852.

Analogous mutant host cell strains have also been produced and are provided by the invention. In particular, the invention provides additional cell strains based on the DB2 mutant strain described above. In one aspect, the invention provides strain DB3, which is based on the tetracycline resistant E. coli strain RR1, and which contains the gyrA462, endA and recA mutations. Hence, strain DB3 may be represented as E. coli RR1 (gyrA462 endA (recA-)). This strain (designated E. coli DB3) is available from Life Technologies and was deposited on Feb. 27, 1999, with the Collection, Agricultural Research Culture Collection (NRRL), 1815 North University Street, Peoria, Ill. 61604 USA as deposit number NRRL B-30097.

In another aspect, the invention provides strain DB3.1, which is identical to strain DB3 (i.e., it is based on E. coil RR1, and contains the gyrA462, endA, and recA mutations) except that DB3.1 is tetracycline sensitive as it does not contain the tetracycline resistance (tet^{r}) gene carried by the other RR1-based strains (RR1, DB1, DB2 and DB3). Strain DB3.1 may therefore be represented as E. coli RR1 (gyrA462 endA (recA-) tet^{s}). This strain (designated E. coil DB3.1) is available from Life Technologies and was deposited on Feb. 27, 1999, with the Collection, Agricultural Research Culture Collection (NRRL), 1815 North University Street, Peoria, Ill. 61604 USA as deposit number NRRL B-30098.

Other mutant host cell strains have also been constructed and are provided by the invention. In this aspect, the mutant host cell strains are based on the tetracycline-resistant E. coil DH10B strain (available commercially from Life Technologies, Inc.). In one such aspect, the invention provides strain DB4, which is a DH10B E. coil strain that carries the gyrA462 mutation and a deletion in the endA gene, as well as carrying the tetracycline resistance transposon Tn10. Strain DB4 thus may be represented as E. coli DH10B (endA Δ(srl-recA)1398::Tn10(tet^{r})). This strain (designated E. coli DB4) is available from Life Technologies and was deposited on Feb. 27, 1999, with the Collection, Agricultural Research Culture Collection (NRRL), 1815 North University Street, Peoria, Ill. 61604 USA as deposit number NRRL B-30106.

In another aspect, the invention provides strain DB5, which is identical to strain DB4 (i.e., it is based on E. coli DH10B, and contains the gyrA462 mutation and the deletion in the endA gene), except that DB5 is tetracycline sensitive as it does not contain the tetracycline resistance (tet^{r}) Tn10 transposon carried by DB4. Strain DB5 thus may be represented as E. coli DH10B (endA Δ(srl-recA)1398). This strain (designated E. coil DB5) is available from Life Technologies and was deposited on Feb. 27, 1999, with the Collection, Agricultural Research Culture Collection (NRRL), 1815 North University Street, Peoria, Ill. 61604 USA as deposit number NRRL B-30107.

Each of these DB mutant host cell strains (DB1, DB2, DB3, DB3.1, DB4, and DB5) are resistant to the effects of expression of the ccdB gene by the host cell. Other mutant host cell strains which contain one or more mutations rendering the host cells resistant to ccdB may also be produced and characterized by the skilled artisan in accordance with the guidance contained herein in combination with information known in the art. In addition, other mutant host cell strains resistant to other toxic genes will also be apparent to one of ordinary skill based on the teachings contained herein and the knowledge in the art, and are encompassed within the scope of the present invention. In one such aspect, these host cell strains of the invention may be mutant cell strains that are resistant to one or more alternative, or one or more additional, toxic genes, including but not limited to kicB, DpnI and other restriction endonucleases, apoptosis-related genes (e.g., ASK1 or members of the bcl-2/ced-9 family), retroviral genes including those of the human immunodeficiency virus (HIV), defensins such as NP-1, inverted repeats or paired palindromic DNA sequences, bacteriophage lytic genes such as those from φX174 (e.g., φX E) or bacteriophage T4, antibiotic sensitivity genes such as rpsL, antimicrobial sensitivity genes such as pheS, plasmid killer genes, eukaryotic transcriptional vector genes that produce a gene product toxic to bacteria such as GATA-1, and the like. Mutant host cell strains that are resistant to such toxic genes may be prepared in accordance with the guidance herein, and may be used in methods of recombinational cloning as detailed herein and in the propagation of nucleic acid molecules or vectors containing the toxic genes which would otherwise be bacteriocidal or bacteriostatic to host cell strains not containing these particular mutations.

Production and Characterization of Mutant Host Cells

The mutant host cells and host cell strains of the invention may be produced by standard mutagenesis methods that will be familiar to one of ordinary skill in the art. For example, to generate an RR1-based mutant host cell strains of the invention (e.g., DB3 and DB3.1), one may obtain an E. coli RR1 host cell strain (e.g. from Life Technologies, Inc, Rockville, Md.) and mutagenize the host cells by any of a number of well-known mutagenesis methods, such as chemical mutagenesis, radiation-induced mutagenesis, and the like. Analogously, to generate a DH10B-based mutant host cell strains of the invention (e.g., DB4 and DB5), one may obtain an E. coli DH10B host cell strain (e.g. from Life Technologies, Inc.; Rockville, Md.) and mutagenize the host cells by any of a number of well-known mutagenesis methods, such as chemical mutagenesis, radiation-induced mutagenesis, and the like.

There are well known procedures for introducing specific mutations into nucleic acid sequences and thus for creating mutant host cell strains containing these specific mutations. A number of these are described in Ausubel, F. M. et al., Current Protocols in Molecular Biology, Wiley Interscience, New York (1989-1996). Mutations can also be designed into oligonucleotides, which can be used to modify existing cloned sequences, or in amplification reactions. Random mutagenesis can also be employed if appropriate selection methods are available to isolate the desired mutant DNA or RNA. Such isolation methods may include, for example, culturing the host cells in culture media (which may be solid or liquid) containing one or more selection agents (such as one or more antibiotics or antimicrobial agents, including tetracycline, ampicillin, kanamycin, chloramphenicol, and the like).

The presence of the desired mutations can be confirmed by isolating the DNA from the mutant host cell strain according to art-known methods such as electrophoretic and chromatographic methods (see, e.g., Ausubel, F. M. et al., Current Protocols in Molecular Biology, Wiley Interscience, New York (1989-1996). Once the DNA has been isolated from the mutant host cells, the specific mutations present in a particular host cell strain may be determined by sequencing the DNA by well known methods, including manual sequencing methods (such as dideoxy sequencing; see Sanger, F., and Coulson, A. R., J. Mol Biol. 94:444-448 (1975); Sanger, F., et al., Proc. Natl. Acad. Sci. USA 74:5463-5467 (1977)) or automated DNA sequencing.

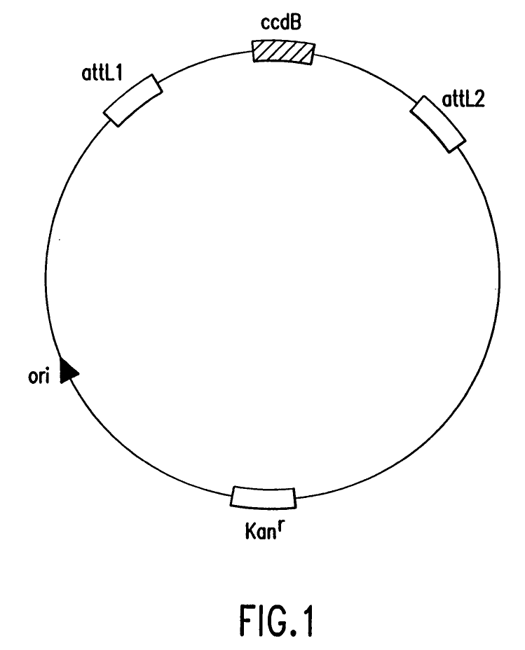

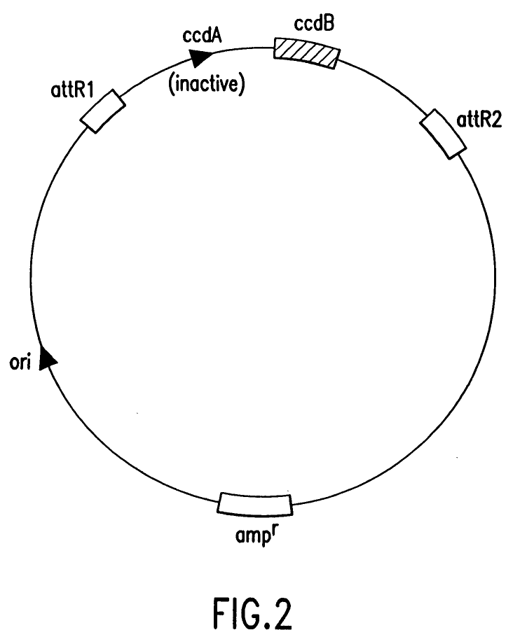

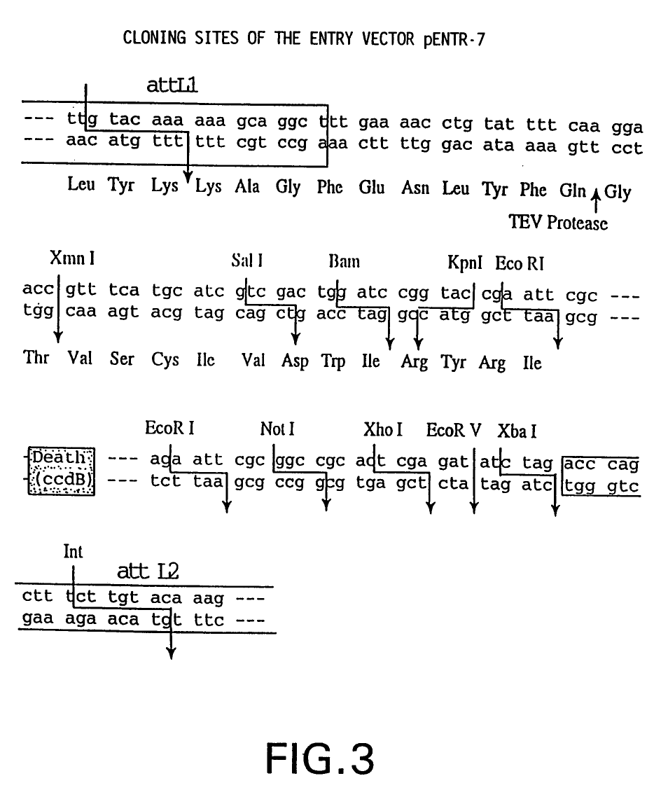

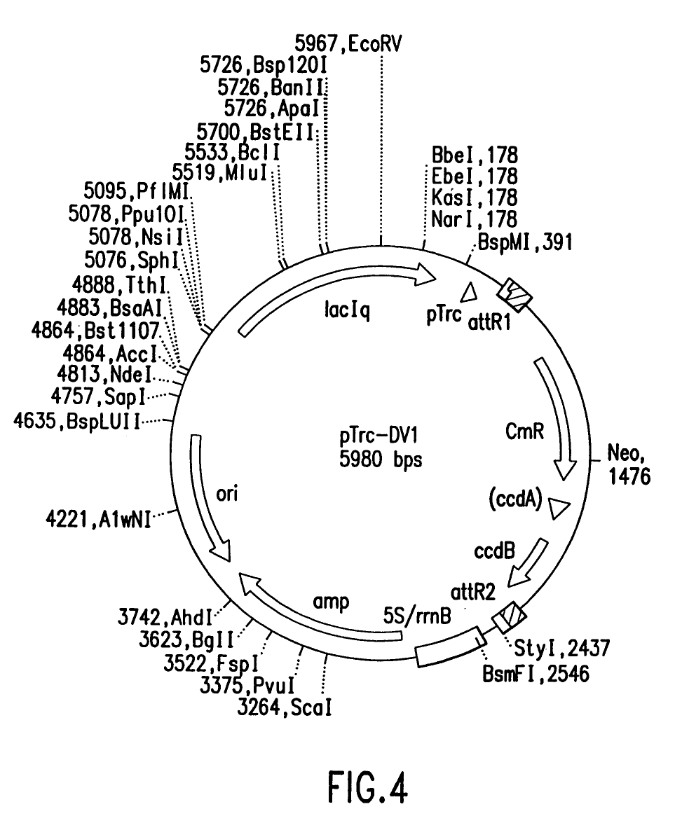

To characterize a host cell or host cell strain to determine its resistance to the presence of a particular toxic gene, several approaches are available. In a first such approach, a genetic construct containing one or more toxic genes (such as those described herein, and particularly ccdB) may be introduced, using any of a number of chemical or physical transformation methods, into the host cells of the invention. For example, one or more Entry Vectors (FIGS. 1, 3) or one or more Destination Vectors (FIGS. 2, 4), available commercially from Life Technologies, Inc., and containing the ccdB gene, may be introduced into the host cell strains of the invention. The transformed host cells may then be cultivated, under conditions favoring the growth of the host cell, in culture medium which may contain one or more selection agents specific for the genetic construct containing the toxic gene. If the host cell strain is able to grow (i.e., form colonies on solid medium, or increase in number or turbidity in liquid culture), the host cell is resistant to the presence of the toxic gene (e.g., the ccdB gene in the example above where one or more Entry Vectors or one or more Destination Vectors are introduced into the host cell) and is said to be a mutant host cell strain of the invention.

In a related method, the resistance of a mutant host cell strain to the presence of one or more toxic genes may be determined by cloning a genetic construct comprising one or more toxic genes, such as those toxic genes described above and particularly ccdB, and subsequently examining the host cells for an increase in copy number of the genetic construct containing the one or more toxic genes. Methods according to this aspect of the invention preferably comprise introducing a genetic construct comprising one or more toxic genes into one or more of the host cells or host cell strains of the invention, and cultivating the host cell or host cell strain under conditions favoring the clonal expansion of the host cell. Following this cultivation, DNA may be isolated as above from the host cells, and the isolated DNA analyzed for an increase in the copy number of the toxic gene.

Kits

In another embodiment, the invention relates to kits comprising one or more of the host cells of the invention. Kits according to this aspect of the invention may comprise a carrier means such as a box, carton, package, drum, or the like, which may be compartmentalized to receive in close confinement therein one or more container means such as tubes, vials, bottles, ampules, packages, envelopes, and the like. The one or more containers may contain one or more host cells of the invention. For example, a first container may contain one or more of the host cell strains of the invention, such as DB3, DB3.1, DB4, or DB5. Additional containers according to this aspect of the invention may comprise one or more components useful in accordance with the application in which the host cells or kits of the invention are to be used, for example one or more genetic constructs (for example, a plasmid, vector, phagemid, cosmid, and the like) containing one or more of the toxic genes described herein (particularly ccdB), one or more buffers or buffer salts, one or more detergents, one or more enzymes (such as one or more recombination proteins, e.g., Int, IHF, or Xis, or combinations thereof, one or more reverse transcriptases, one or more nucleic acid polymerases, or one or more restriction enzymes), one or more nucleotides (which may be detectably labeled, as with a fluorophore, a chromophore, an enzyme, or a radioisotope), one or more proteins (such as albumin, one or more ribosomal proteins, and the like), one or more selection agents (e.g., one or more antibiotics, detergents, dyes, antimicrobial agents, and the like), and/or one or more culture media or components thereof suitable for cultivation of the host cells of the invention.

Uses of Host Cells

The mutant host cells and host cell strains of the invention may be used for a variety of purposes. For example, the mutant host cells may be used to clone genetic constructs (e.g., nucleic acid molecules (which may be linear or circular), vectors, plasmids, phagemids, cosmids, and the like) containing one or more of the toxic genes described herein, particularly ccdB. Methods according to this aspect of the invention may comprise multiple steps, for example introducing a genetic construct comprising one or more toxic genes into one or more of the host cells or host cell strains of the invention, and cultivating the host cell or host cell strain under conditions favoring the clonal expansion of the host cell. As used herein, “conditions favoring the clonal expansion of the host cell” means the optimal incubation conditions (including optimal nutritional, physical (e.g., temperature, light, humidity, etc.), and chemical conditions) that provide for the most rapid and healthy growth of the host cell strain being cultivated. As a practical matter, and as one of ordinary skill will be aware, growth of a particular host cell strain may be determined by plating cultivation fluid containing the host cell onto solid culture media, incubating for an appropriate period of time, and counting colonies that develop, with a higher number of colonies indicating more optimal growth conditions. Analogously, as one of ordinary skill will also be aware, growth of a particular host cell strain may be determined by inoculating the host cell into liquid culture media, incubating for an appropriate period of time, and determining the turbidity of the culture media (e.g., by spectrophotometry), with a higher turbidity indicating more optimal growth conditions. Mutant host cells of the invention will be resistant to the one or more toxic genes carried by the genetic constructs with which they have been transformed, and the genetic constructs containing the toxic genes will be replicated as the host cells grow. Hence, an increase in copy number of genetic constructs containing toxic genes may be accomplished using the host cells and host cell strains of the invention.

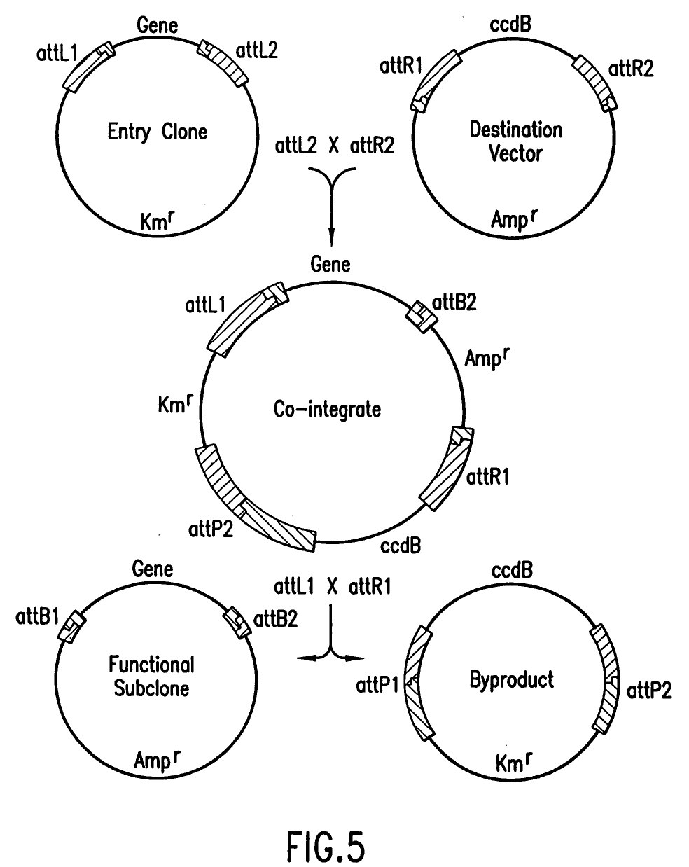

In another application, the host cells and host cell strains of the invention may be used in methods of recombinational cloning, whereby segments of nucleic acid molecules of interest or populations of such molecules are exchanged, fused, inserted, replaced, substituted or modified, in vitro or in vivo without the use of restriction enzymes. Such methods of recombinational cloning are generally depicted in FIG. 5, wherein an Entry Clone containing a gene of interest, flanked by attL1 and attL2 sites, is combined with a Destination Vector containing the ccdB gene flanked by attR1 and attR2 sites. Upon incubation, the attL1 and attR1 sites and the attL2 and attR2 sites recombine to create a functional subclone (which may be an expression vector, for example) and a by-product plasmid. Methods and applications for recombinational cloning are provided in detail in commonly owned, co-pending U.S. application Ser. No. 08/486,139, filed Jun. 7, 1995 (now abandoned), Ser. No. 08/663,002, filed Jun. 7, 1996 (now U.S. Pat. No. 5,888,732), Ser. No. 09/005,476, filed Jan. 12, 1998, Ser. No. 09/177,387, filed Oct. 23, 1998, Ser. No. 09/233,492, filed Jan. 20, 1999, Ser. No. 09/233,493, filed Jan. 20, 1999, No. 60/122,389, filed Mar. 2, 1999, No. 60/126,049, filed Mar. 23, 1999, No. 60/136,744, filed May 28, 1999, Ser. No. 09/296,280, filed Apr. 22, 1999, Ser. No. 09/296,281, filed Apr. 22, 1999, Ser. No. 09/432,085, filed Nov. 2, 1999, and Ser. No. 09/438,358, filed Nov. 12, 1999, the disclosures of all of which are incorporated by reference herein in their entireties.

It will be understood by one of ordinary skill in the relevant arts that other suitable modifications and adaptations to the methods and applications described herein are readily apparent and may be made without departing from the scope of the invention or any embodiment thereof. Having now described the present invention in detail, the same will be more clearly understood by reference to the following examples, which are included herewith for purposes of illustration only and are not intended to be limiting of the invention.

### Examples

### Example 1

### Preparation and Characterization of Mutant Host Cells

The mutant host cells and host cell strains of the invention were produced by standard mutagenesis methods that will be familiar to one of ordinary skill in the art. For example, to generate an RR1-based mutant host cell strains of the invention (e.g., DB3 and DB3.1), E. coli RR1 host cell strains were obtained from Life Technologies, Inc. and mutagenized by chemical mutagenesis or radiation-induced mutagenesis. Analogously, to generate a DH10B-based mutant host cell strains of the invention (e.g., DB4 and DB5), E. coli DH10B host cells were obtained from Life Technologies, Inc. and mutagenized in the same fashion as for the RR1-based host cells. Specific mutations were introduced as described in Ausubel, F. M. et al., Current Protocols in Molecular Biology, Wiley Interscience, New York (1989-1996).

The presence of the desired mutations was confirmed by isolating the DNA from the mutant host cell strain according to art-known methods such as electrophoretic and chromatographic methods (see, e.g., Ausubel, F. M. et al, Current Protocols in Molecular Biology, Wiley Interscience, New York (1989-1996). Once the DNA was isolated from the mutant host cells, the specific mutations present in a particular host cell strain were determined by sequencing the DNA by well known methods (see Sanger, F., and Coulson, A. R., J. Mol Biol. 94:444-448 (1975), Sanger, F., et al., Proc. Natl. Acad. Sci. USA 74:5463-5467 (1977)) or by automated DNA sequencing.

To characterize a host cell or host cell strain to determine its resistance to the presence of a particular toxic gene, several approaches were carried out. In a first such approach, a genetic construct containing one or more toxic genes (such as those described herein, and particularly ccdB) was introduced, using any of a number of chemical or physical transformation methods, into the host cells of the invention. For example, one or more Entry Vectors (FIGS. 1, 3) or one or more Destination Vectors (FIGS. 2, 4), available commercially from Life Technologies, Inc., and containing the ccdB gene, were introduced into the host cell strains of the invention: The transformed host cells were be cultivated, under conditions favoring the growth of the host cell, in culture medium which may contain one or more selection agents specific for the genetic construct containing the toxic gene. If the host cell strain was able to grow (i.e., form colonies on solid medium, or increase in number or turbidity in liquid culture), the host cell was said to be resistant to the presence of the toxic gene (e.g., the ccdB gene in the example above where one or more Entry Vectors or one or more Destination Vectors are introduced into the host cell) and was said to be a mutant host cell strain of the invention.

In a related method, the resistance of a mutant host cell strain to the presence of one or more toxic genes was determined by cloning a genetic construct comprising one or more toxic genes, such as those toxic genes described above and particularly ccdB, and subsequently examining the host cells for an increase in copy number of the genetic construct containing the one or more toxic genes.

### Example 2

### Characterization of Strain DB3

RR1 E. coli host cells were mutagenized, and DNA isolated from the host cells, as described in Example 1 above, to generate strain DB3. Upon sequencing the isolated DNA, strain DB3 was found to contain the gyrA462 and endA mutations, and a complete deletion of the recA gene. Hence, strain DB3 was represented as E. coli RR1 (gyrA462 endA (recA-)).

This strain (designated E. coli DB3) is available from Life Technologies and was deposited on Feb. 27, 1999, with the Collection, Agricultural Research Culture Collection (NRRL), 1815 North University Street, Peoria, Ill. 61604 USA as deposit number NRRL B-30097.

### Example 3

### Characterization of Strain DB3.1

DB3 E. coli host cells (Example 2) were mutagenized, and DNA isolated from the host cells, as described in Example 1 above, to generate strain DB3.1. Upon sequencing the isolated DNA, strain DB3.1 was found to contain the same gyrA462, endA, and recA mutations as DB3, and to be tetracycline sensitive due to deletion of the tet^{r} gene. Hence, strain DB3.1 was represented as E. coli RR1 (gyrA462 endA (recA-) tet^{s}).

This strain (designated E. coli DB3.1) is available from Life Technologies and was deposited on Feb. 27, 1999, with the Collection, Agricultural Research Culture Collection (NRRL), 1815 North University Street, Peoria, Ill. 61604 USA as deposit number NRRL B-30098.

### Example 4

### Characterization of Strain DB4

DH10B E. coil host cells (Life Technologies, Inc.; Rockville, Md.) were mutagenized, and DNA isolated from the host cells, as described in Example 1 above, and the mutated cells were transformed with the tetracycline resistance transposon Tn10 to generate strain DB4. Upon sequencing the isolated DNA, strain DB4 was found to contain the gyrA462 and endA mutations, a deletion at base 1398 of the recA gene, and the Tn10 transposon. Hence, strain DB4 was represented as E. coli DH10B (gyrA462 endA Δ(srl-recA)1398::Tn10(tet^{r})).

This strain (designated E. coli DB4) is available from Life Technologies and was deposited on Feb. 27, 1999, with the Collection, Agricultural Research Culture Collection (NRRL), 1815 North University Street, Peoria, Ill. 61604 USA as deposit number NRRL B-30106.

### Example 5

### Characterization of Strain DB5

DH10B E. coli host cells (Life Technologies, Inc.; Rockville, Md.) were mutagenized, and DNA isolated from the host cells, as described in Examples 1 and 4 above, except that the mutated cells were not transformed with the tetracycline resistance transposon Tn10, to generate strain DB5. Upon sequencing the isolated DNA, strain DB5 was found to contain the same gyrA462, endA and recA mutations as DB4, and to be tetracycline sensitive. Hence, strain DB5 was represented as E. coli DH10B (gyrA462 endA Δ(srl-recA)1398 tet^{s}).

This strain (designated E. coli DB5) is available from Life Technologies and was deposited on Feb. 27, 1999, with the Collection, Agricultural Research Culture Collection (NRRL), 1815 North University Street, Peoria, Ill. 61604 USA as deposit number NRRL B-30107.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A mutant host cell containing a gyrA gene, an endA gene, and a recA gene, wherein said gyrA and endA genes contain one or more mutations that render said host cell resistant to the expression of one or more toxic genes.

2. The mutant host cell of claim 1, further comprising one or more mutations in said recA gene.

3. The mutant host cell of claim 1, further comprising one or more mutations in one or more additional genes which render said host cell resistant to the expression of two or more toxic genes.

4. The mutant host cell of claim 1 or claim 2, further comprising one or more genetic elements that enable said mutant host cell to grow on tetracycline-containing culture media.

5. The mutant host cell strain of claim 1 or claim 2, wherein said mutation in said gyrA gene is gyrA462.

6. The mutant host cell strain of claim 1, wherein said mutation in said recA gene is Δ(srl-recA)1398.

7. The mutant host cell of claim 4, wherein said genetic element is a tetracycline resistance gene or transposon Tn10.

8. The mutant host cell of claim 1, wherein said host cell is an Escherichia coli cell.

9. The mutant host cell of claim 1, wherein said toxic gene is selected from the group consisting of ccdB, kicB, DpnI, an apoptosis-related gene, a retroviral gene, a defensin, a bacteriophage lytic gene, ΦX E, an antibiotic sensitivity gene, an antimicrobial sensitivity gene, a plasmid killer gene, and a eukaryotic transcriptional vector gene that produces a gene product toxic to bacteria.

10. The mutant host cell of claim 1, wherein said toxic gene is ccdB.

11. The mutant host cell of claim 1, wherein said host cell is selected from the group consisting of a DB3 cell (deposit number NRRL B-30097), a DB3.1 cell (deposit number NRRL B-30098), a DB4 cell (deposit number NRRL B-30106), and a DB5 cell (deposit number NRRL B-30107), or a mutant or derivative thereof.

12. Mutant host cell strain DB3 (deposit number NRRL B-30097).

13. Mutant host cell strain DB3.1 (deposit number NRRL B-30098).

14. Mutant host cell strain DB4 (deposit number NRRL B-30106).

15. Mutant host cell strain DB5 (deposit number NRRL B-30107).

16. A method of cloning a genetic construct comprising one or more toxic genes, said method comprising introducing said genetic construct into the host cell of claim 1 or claim 2 and cultivating said host cell under conditions favoring the clonal expansion of said host cell.

17. The method of claim 16, wherein said toxic gene is selected from the group consisting of ccdB, kicB, DpnI, an apoptosis-related gene, a retroviral gene, a defensin, a bacteriophage lytic gene, ΦX E, an antibiotic sensitivity gene, an antimicrobial sensitivity gene, a plasmid killer gene, and a eukaryotic transcriptional vector gene that produces a gene product toxic to bacteria.

18. The method of claim 16, wherein said toxic gene is ccdB.

19. The method of claim 15, wherein said host cell is selected from the group consisting of a DB3 cell (deposit number NRRL B-30097), a DB3.1 cell (deposit number NRRL B-30098), a DB4 cell (deposit number NRRL B-30106), and a DB5 cell (deposit number NRRL B-30107), or a mutant or derivative thereof.

20. A kit comprising one or more of the mutant host cells of claim 1 or claim 2.

21. The kit of claim 20, further comprising one or more additional components selected from the group consisting of one or more culture media suitable for cultivation of said host cell, one or more selection agents, one or more genetic constructs comprising one or more toxic genes, one or more enzymes, one or more nucleotides, one or more buffers, and the like.

22. The kit of claim 19, wherein said host cell is selected from the group consisting of a DB3 cell (deposit number NRRL B-30097), a DB3.1 cell (deposit number NRRL B-30098), a DB4 cell (deposit number NRRL B-30106), and a DB5 cell (deposit number NRRL B-30107), or a mutant or derivative thereof.


SEQUENCE LISTING (2 sequences)

SEQ ID NO: 1 - DNA, 120 bp, Artificial Sequence
  Cloning sites of the synthetic pENTR-7 Entry Vector
ttgtacaaaaaagcaggctttgaaaacctgtattttcaaggaaccgtttcatgcatcgtcgactggatccggtaccgaattcgcagaattcgcggccgcactcgagatatctagacccag

SEQ ID NO: 2 - protein, 27 aa, Artificial Sequence
  Predicted translation product of cloning sites of the synthetic pENTR-7 Entry Vector
LYKKAGFENLYFQGTVSCIVDWIRYRI